(19) United States
(12) Patent Application Publication
(10) Pub. No.: US 2013/0281997 A1
(43) Pub. Date: Oct. 24, 2013

(54) METHOD FOR TREATMENT OF VT USING ABLATION

(71) Applicant: Scott W. DAVIE, Beaconsfield (CA)

(72) Inventor: Scott W. DAVIE, Beaconsfield (CA)

(73) Assignee: MEDTRONIC CRYOCATH LP, Toronoto (CA)

(21) Appl. No.: 13/451,637

(22) Filed: Apr. 20, 2012

(51) Int. Cl. A61B 18/02; A61B 18/18; A61B 18/04

(52) U.S. Cl. 606/21; 606/32; 606/33

(57) ABSTRACT

A method and system for treating ventricular tachycardia (VT) by ablating scarred myocardial tissue containing a reentrant VT circuit. The method generally includes measuring electrical activity at a plurality of sites within a heart, identifying an area of scarred myocardial tissue based at least in part on the electrical activity measurements, and ablating substantially all of the scarred area. The system generally includes a medical device having a distal end, a plurality of electrical conduction sensors coupled to the distal end, and a console in communication with the distal end of the device, the console including a computer with display. The computer may be programmed to identify optimal ablation sites within a target tissue and the location of an isthmus associated with a reentrant VT circuit within a target tissue, the identifications being based at least in part on the measurements of the electrical conduction sensors.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for cardiovascular rhythm management, including the treatment of ventricular tachycardia.

### BACKGROUND OF THE INVENTION

Ventricular tachycardia (VT) is a heart rhythm faster than about 100 beats per minute that arises distal to the bundle of His (usually within the ventricles). VT may result from ischemic or structural heart disease or electrolyte deficiencies, and can be triggered by the use of certain drugs, ingestion of digoxin, or from certain systemic diseases (such as rheumatoid arthritis, sarcoidosis, and systemic lupus), structural congenital disorders, or prior myocardial infarction. A myocardial infarction is the death of myocardial cells, usually as a result of oxygen deprivation of the cells. After an infarction occurs, the cardiomyocytes in the infracted myocardium are replaced by fibrous scar tissue. This scar tissue is primarily composed of myofibroblasts and collagen-rich extracellular matrix, and the tissue remains metabolically active. However, myocardial scar tissue hinders systolic and diastolic function of the heart by stiffening otherwise pliable myocardial tissue. Further, scar tissue reduces electrical conduction between cardiomyocytes and impairs impulse propagation.

If VT is due to myocardial infarction, the abnormal heart beats are usually caused by “reentry circuit” in the border zone of or within the infarct or scarring. Scarred areas (regions of functional block), although themselves impairing or preventing proper electrical propagation, may contain within them isthmuses or channels of slowed electrical conduction. These isthmuses, in turn, may perpetuate an endless electrical loop or circuit that produces a rapid and possibly irregular heartbeat. This loop is often referred to as a “reentrant VT circuit.”

VT may be treated using catheter ablation. Standard procedures for treating a reentrant VT circuit include mapping areas of the heart to locate a critical isthmus. The isthmus is then ablated to destroy the reentry circuit. However, isthmus identification has proven to be difficult, tedious, and unreliable. For example, many VTs are unmappable using electrocardiography because of hemodynamic instability or poor reproducibility. For these VTs, other mapping methods, such as pace mapping, may be used to locate a critical isthmus and sometimes also the entry and exit points of some types of VTs (such as focal VT or frequent symptomatic premature ventricular complexes). Often, multiple mapping methods are used to complement each other, such as pace mapping, activation mapping, entrainment mapping, and substrate mapping. Unfortunately, not only can the use of multiple tests be costly and time-consuming, but even the use of a variety of tests does not always produce a reliable and useful identification of critical ablation sites. Finally, very few medical practitioners are qualified to perform these techniques, which can inflate treatment costs.

The system and method described herein are directed to the destruction of a reentrant VT circuit that does not necessitate the complex mapping procedures required to locate the isthmus. Also, the system and method may completely disrupt the reentry mechanism within myocardial scarring in one treatment. Further, the system and method may result in a minimal amount to no damage of healthy myocardial tissue.

### SUMMARY OF THE INVENTION

The present invention advantageously provides a method and system for treating VT by ablating scarred myocardial tissue containing a reentrant VT circuit. The method includes identifying an area of scarred myocardial tissue based at least in part on a measurement of electrical activity at a plurality of sites within a heart. The electrical activity may be measured by a plurality of electrodes coupled to a mapping device. Once an area of scarred myocardial tissue and its border are identified, substantially all of the scarred area is ablated using an ablation device capable of radiofrequency ablation (including phased radiofrequency ablation techniques), ultrasound ablation, microwave ablation, laser ablation, hot balloon ablation, and/or cryoablation. Epicardial and/or endocardial tissue may be ablated. Further, mapping and ablation functions may be performed by the same medical device. The method may further include displaying a visual depiction of the electrophysiological anatomy of the scarred myocardial tissue on a display device in electrical communication with a computer. The computer may be programmed to identify optimal ablation sites, such as an isthmus associated with a reentrant circuit, based at least in part on the measurement of the plurality of sensors.

The system may include a medical device with an elongate body, a proximal end, a distal end, and a plurality of electrical conduction sensors coupled to the distal end, a console in communication with the distal end of the device including a computer, a display, a cryogenic fluid reservoir, and/or radiofrequency generator. The computer may be programmed to identify optimal ablation sites on a target tissue and the location of an isthmus associated with a reentrant VT circuit within a target tissue, the identification based at least in part on the measurement of the electrical conduction sensors. The device may further include a treatment element and/or a plurality of electrodes at the distal end.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a flow chart of a method for identifying and ablating scarred myocardial tissue;

FIG. 2A shows a method for identifying a myocardial scar boundary;

FIG. 2B shows a method for ablating the scarred myocardial tissue;

FIG. 3 shows a system for identifying and ablating scarred myocardial tissue;

FIG. 4 shows a mapping focal catheter;

FIG. 5 shows a mapping catheter having an expandable element;

FIG. 6 shows a radiofrequency ablation catheter;

FIG. 7 shows a cryoablation catheter having an expandable element;

FIG. 8 shows a combined radiofrequency ablation catheter and cryoablation catheter; and

FIG. 9 shows a combined ablation catheter and mapping catheter.

### DETAILED DESCRIPTION OF THE INVENTION

As used herein, the term “scar,” “scarred myocardial tissue,” or “scar tissue” refers to an area of heart tissue in which cardiomyocytes (cardiac muscle cells) are replaced by tissue composed of fibroblasts (such as myofibroblasts) and extracellular matrix. In general, this fibrous scar tissue may be identified as an area of stiffened tissue and of decreased or non-existent electrical impulse propagation (impaired electrical coupling between cardiomyocytes).

As used herein, the term “ablated tissue,” “ablation lesion,” or the like refers to an area of heart tissue that has been treated using an ablation treatment device (for example, a radio frequency ablation or cryoablation catheter). Ablation of the myocardium results in death of cardiomyocytes and the subsequent formation of scar tissue.

As used herein, the term “normal” or “healthy” tissue refers to mammalian biological tissue that is unaffected by disease or congenital problems and does not comprise scar tissue.

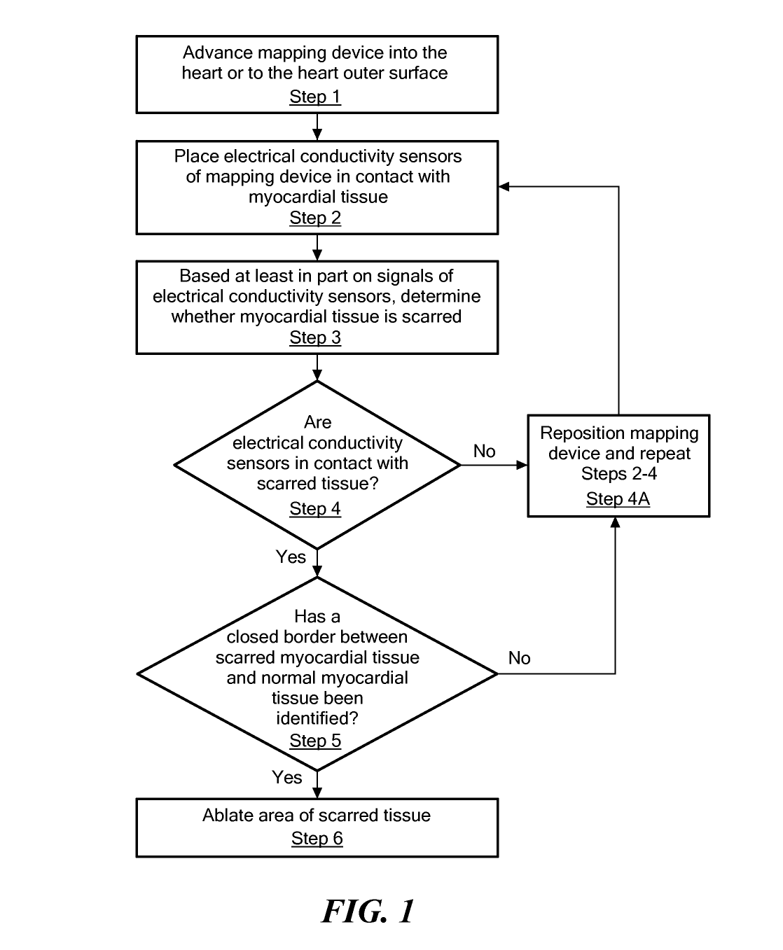

Referring now to FIG. 1, a flow chart of a method for identifying and ablating scarred myocardial tissue 10 is shown. The method generally includes measuring electrical activity at a plurality of sites within a heart, identifying an area of scarred myocardial tissue 10 based at least in part on the electrical activity measurements, and ablating substantially all of the scarred area 10. Because an area of scarred tissue 10 frequently contains an isthmus 11 of slow electrical conduction within, ablating substantially all of the scar tissue is likely to result in ablation of the isthmus 11. As a result, the reentrant VT circuit is destroyed.

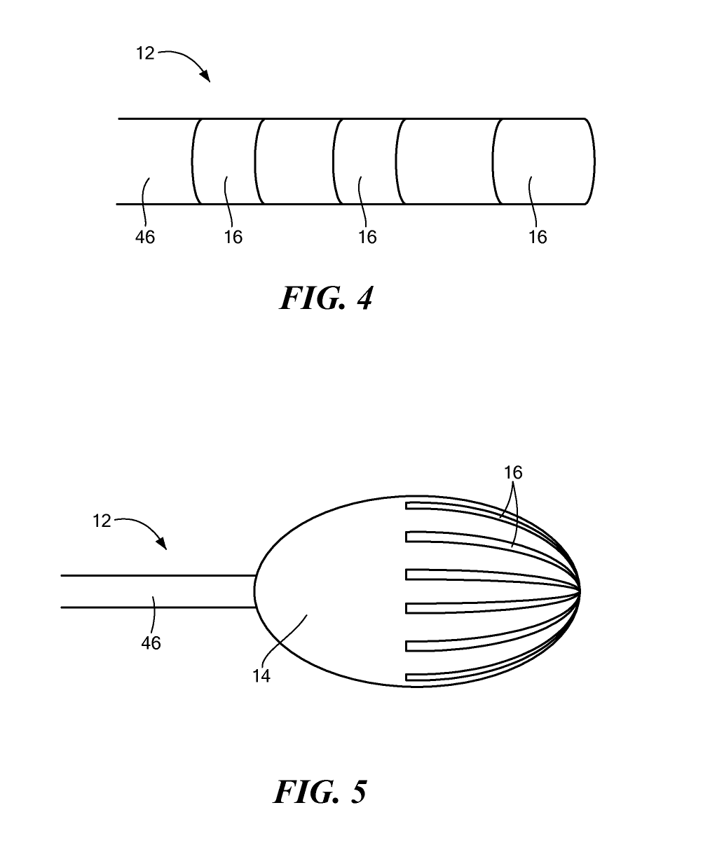

To identify an area of scarred myocardial tissue 10, a mapping device 12 is positioned endocardially or epicardially, such as by advancing the device 12 through the patient's vasculature and into the heart or through a thoracic incision and into the pericardial space (Step 1). The mapping device 12 may be a focal catheter (as shown in FIG. 4) or catheter with an expandable element 14 (as shown in FIG. 5) that includes a plurality of sensors 16. Further, the mapping device 12 and ablation device 18 may be combined within the same device 20 (as shown in FIGS. 9 and 10).

The sensors 16 detect electrical activity in the heart as the myocardial cells polarize and depolarize. The sensors 16 may be, for example, electrodes capable of detecting electrocardiographic measurements (electrocardiograms). As used herein, the term “electrode” and “sensor” may be used interchangeably when referring to an element used only for mapping or for ablation (such as radiofrequency (RF) or PRF) and mapping. An element used only for ablation is referred to as “electrode” 22. As the mapping device 12 (for example, a catheter) is put in contact with various areas of the heart, such as be dragging the catheter along the endocardium or epicardium or repositioning the mapping catheter 12 at different locations within or on the heart (Steps 2 and 4A), the electrical activity of each area is measured, a technique called voltage mapping. Scarred myocardial tissue 10 is generally associated with areas of low voltage (typically less than approximately 0.1 mV), and therefore scarred tissue 10 may be identified using voltage mapping (Steps 3 and 4). Any type of navigation and/or mapping system may be used for this purpose.

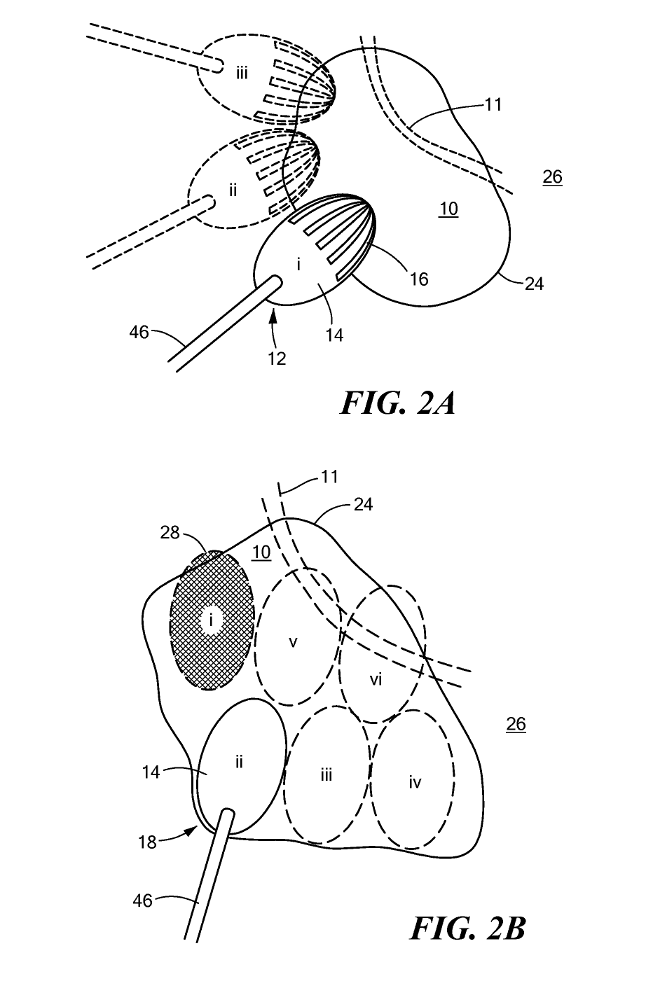

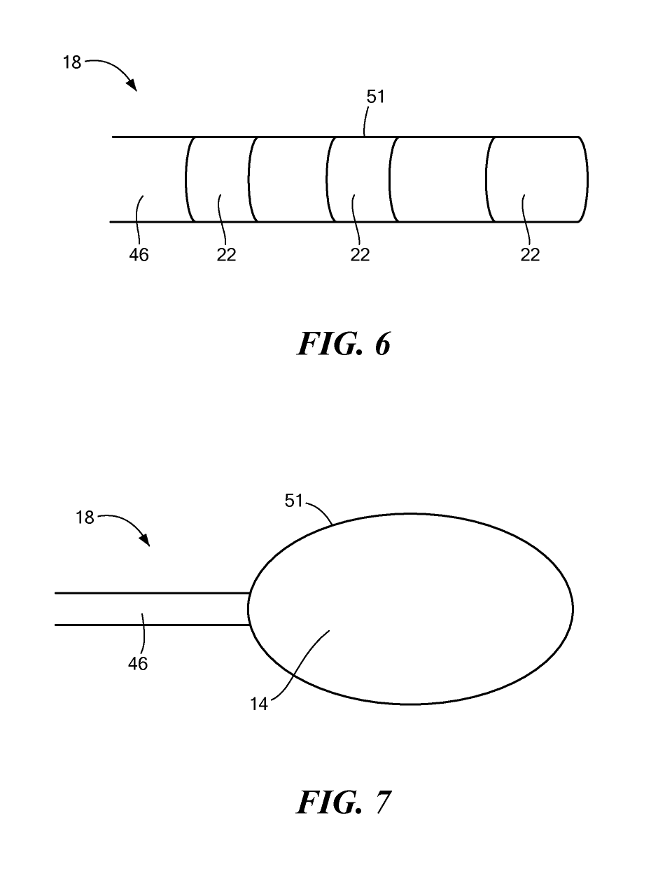

Continuing to refer to FIG. 1, an area of scarred tissue 10 is identified using voltage mapping. Specifically, an enclosed border 24 between the scarred myocardial tissue 10 and healthy myocardial tissue 26 is identified (Step 5). Isthmuses 11 of slow electrical conductivity are likely to exist in or adjacent areas of scarred myocardial tissue 10. Therefore, the entire border 24 encircling the scarred tissue 10 is identified in Step 5 (also as shown in FIG. 2A). The mapping catheter 12 will have to be relocated to several different positions to completely map the entire border 24 (Step 4A). Once the area of scar tissue 10 and border 24 thereof is identified, an ablation catheter 18 (as shown in FIG. 6 or 7) or a medical device having both ablation and mapping functions 20 (as shown in FIG. 9) is used to ablate substantially all of the scar tissue 10 (Step 6). The ablated area 28 may include at least a portion of the border 24 between scarred 10 and healthy 26 myocardial tissue. The object of the present method is to destroy the reentrant VT circuit within an area of scarred myocardial tissue 10 with a few applications of ablative energy, instead of necessitating extensive mapping of an isthmus within the scarred area followed by repeated ablation, induction, and further mapping. Therefore, a substantial portion of the scarred area 10 will be ablated to increase the likelihood of reentrant VT circuit destruction (as shown in FIG. 2B). For example, between approximately 75% to 100% of the scarred area 10 may be ablated. Instead of using multiple mapping methods to locate an isthmus 11 for ablation, the present method only uses a technique such as voltage mapping to identify an area of scarred myocardial tissue 10 and the border 24 thereof. These scarred areas of tissue 10, which are likely to include an isthmus 11 that is the source of a reentrant VT circuit, are then ablated. Consequently, scar tissue is formed in the area of the ablated isthmus 11, thereby blocking the abnormal electrical impulses from being conducted and destroying the reentrant VT circuit. Thus, the present method greatly reduces the time and complexity of performing ablation treatment of VT with respect to existing mapping and ablation techniques.

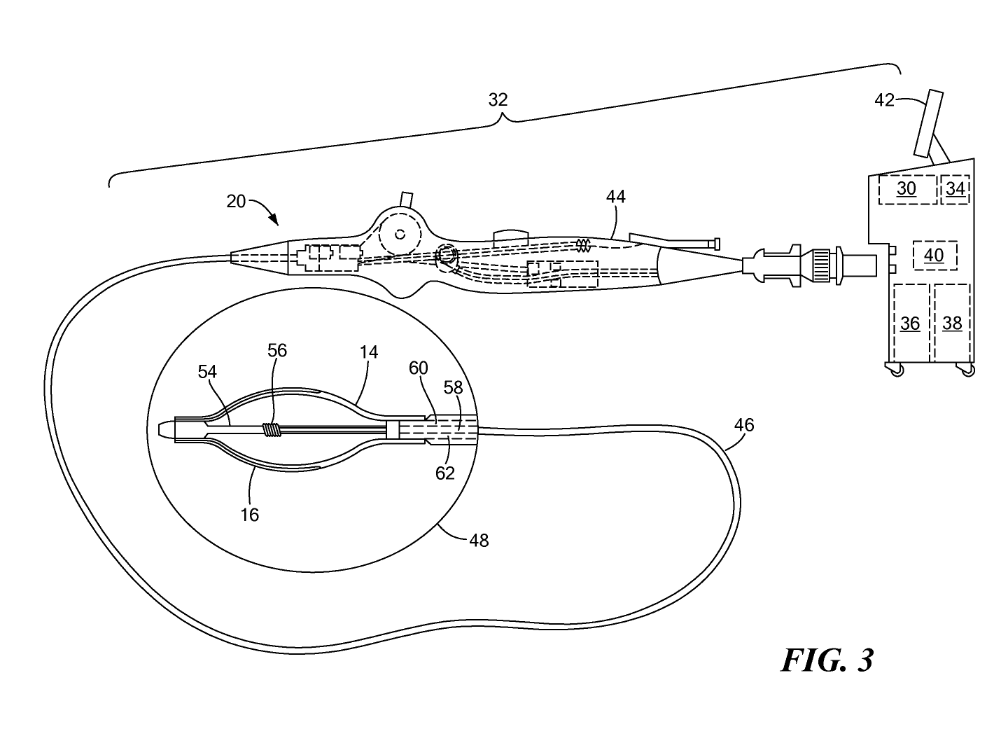

Referring now to FIGS. 2A and 2B, methods of identifying a scar boundary 24 and ablating scarred myocardial tissue 10 are shown. The method of FIG. 1 generally includes measuring electrical activity at a plurality of sites within a heart, identifying an area of scarred myocardial tissue 10 based at least in part on the electrical activity measurements, and ablating the scarred area 10. FIG. 2A shows the scar mapping step of the method described in FIG. 1 in more detail. Specifically, a mapping catheter 12 (or a device having both mapping and ablation functionality 20) is positioned at a first location i and electrical activity is measured. The mapping catheter 12, 20 is then positioned at a second location ii to measure electrical activity, and so on until the entire scar border 24 is identified (a third location iii is also shown in FIG. 2A). Alternatively, the mapping catheter 12, 20 may be dragged along the endocardium or epicardium without disrupting contact between the tissue and the sensors 16. Further, a computer 30 in communication with the mapping catheter 12, 20 (as described in FIG. 3) may be programmed to extrapolate an entire scar border 24 if less than the entire border is mapped by the mapping catheter 12, 20.

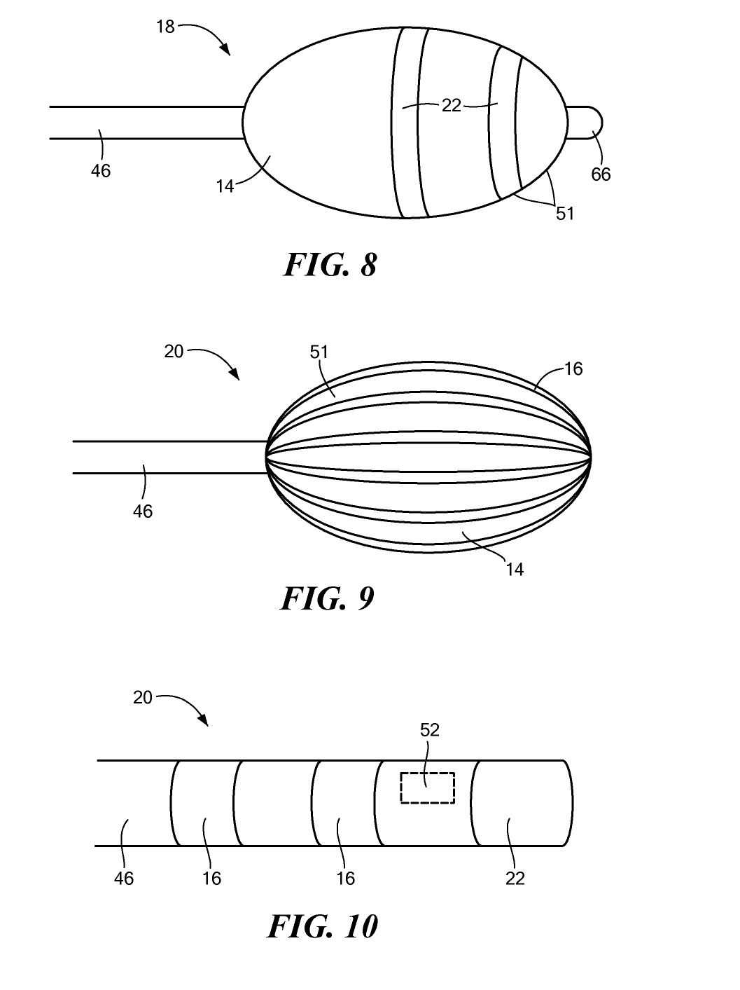

FIG. 2B shows the ablation step of the method described in FIG. 1 in more detail. Specifically, an ablation catheter (for example, an ablation catheter 18 having an expandable element 14, as shown in FIGS. 7 and 8, or a combination ablation and mapping device 20 having a balloon, as shown in FIG. 9) is positioned within the scar boundary 24 in contact with the scarred myocardial tissue 10 (at a first location i) and activated. The ablation device 18 (or combination ablation and mapping device 20) is placed in contact with the tissue in any manner that best suits the ablation device 18, 20 used and the area of treatment. For example, an ablation device 18, 20 having an expandable element 14 (as shown in FIG. 2B) may be in contact with the tissue at the distal end of the expandable element 14 or in contact along an area of the circumference of the expandable element 14. After a sufficient ablation time at the first position, the ablation catheter 18, 20 is moved to a second location ii and activated. This step is repeated as many times as is necessary to ablate substantially the entire area of scarred myocardial tissue 10 (for example, between approximately 75% and 100% of the scarred area 10). For example, FIG. 2B shows repetition of the ablation process in six different locations, which are given reference numbers i, ii, iii, iv, v, and vi. The effective ablation time may be determined using known methods, such as based on the desired ablation depth and known ablation characteristics of the ablation catheter 18, 20. Further, a position at which the ablation catheter 18, 20 is positioned may overlap the scar boundary 24 or an area that has already been ablated 28. Although healthy myocardial tissue 26 proximate the scar boundary 24 may be ablated to completely destroy some reentrant VT circuits, the procedure should be performed to prevent as little damage to healthy tissue 26 as possible.

Referring now to FIG. 3, a system 32 for identifying and ablating scarred myocardial tissue 10 is shown. The system generally includes a device 12 for mapping an area of tissue, a device for treating tissue 18, and a console 34 that houses various system controls. The mapping device 12 and ablation device 18 may be separate devices or combined into a single device 20 with mapping and ablation functionality. The system 32 may be adapted for radiofrequency (RF) ablation and/or PFA (as shown in FIG. 6), cryoablation (as shown in FIG. 7), or both (as shown in FIGS. 8, 9, and 10), or other ablation methods such as laser ablation, microwave ablation, hot balloon ablation, or ultrasound ablation (not shown). Further, the device may have both mapping and ablation functionality 20 (as shown in FIGS. 9 and 10). The console 34 may include one or more of a fluid (such as coolant or saline) reservoir 36, fluid return reservoir 38, energy generator 40 (for example, an RF generator), and computer 30 with display 42, and may further include various other displays, screens, user input controls, keyboards, buttons, valves, conduits, connectors, power sources, and computers for adjusting and monitoring system parameters.

Continuing to refer to FIG. 3, the computer 30 may be programmable to use time/amplitude morphology data to identify optimal ablation sites within the heart and to determine the location of an isthmus 11 based on voltage mapping data (such as electrical conductivity, signal amplitudes, and monophasic action potentials). For example, the mapping device 12, 20 may measure signal amplitudes, relative activation time, activation duration, monophasic action potential, and other measurements that generally describe time/amplitude morphology of normal and/or reentrant circuits. The computer 30 is programmed with an algorithm to extrapolate wave front direction from the time/amplitude morphology data from multiple electrodes. Further, for example, using the known diameter of the ablation device 18, 20 and the identified location and area of scarred tissue 10, the computer 30 may calculate the optimal number and position of ablation sites. Still further, the computer display 42 (and/or other displays included in the system) may show a visualization of the heart including areas of scarred myocardial tissue 10 and the boundaries 24 between scarred tissue 10 and healthy tissue 26. Referencing a displayed image and/or voltage data, the user may identify the scar boundary 24 and thus the area of scarred tissue 10 targeted for ablation.

Continuing to refer to FIG. 3, the device with mapping functionality 12 may be a mapping catheter generally including a handle 44 and an elongate body 46 having a distal end 48 and a proximal end 50, and one or more mapping elements or sensors 16. The mapping device 12 may be a catheter including an expandable element 14 (such as a balloon or wire mesh) at the distal end (as shown in FIG. 5) or the mapping device 12 may be a focal catheter (as shown in FIG. 4). The mapping elements are sensors 16 or electrodes capable of sensing electrical activity within the myocardial cells as the cells polarize and depolarize. Further, the mapping elements 16 may include one or more temperature, pressure, magnetic, or other sensors.

Continuing to refer to FIG. 3, the ablation device 18 may be an ablation catheter generally including a handle 44, an elongate body 46 having a distal end 48 and a proximal end 50, and one or more treatment elements 51. The handle 44 may include various knobs, levers, user control devices, input ports, outlet ports, connectors, lumens, and wires. The one or more treatment elements 51 may be expandable elements 14 such as balloons (as shown in FIGS. 2, 7, 8, 9), such as used for cryoablation or hot balloon ablation (for example, Toray Satake Balloon®), or the one or more treatment elements 51 may be electrodes 22 (as shown in FIG. 6), such as used for RF and PRF ablation. Further, the ablation catheter 18 may include one or more thermoelectric cooling elements 52 (as shown in FIG. 10). The elongate body 46 may further include one or more lumens. If the ablation catheter 18 is a cryoablation catheter, for example, the elongate body 46 may include a shaft 54, a fluid injection element 56, a main lumen 58, a fluid injection lumen 60 in fluid communication with the coolant reservoir, and a fluid return lumen 62 in fluid communication with the coolant return reservoir. In some embodiments, one or more other lumens may be disposed within the main lumen 58, and/or the main lumen 58 may function as the fluid injection lumen 60 or the fluid return lumen 62. If the ablation catheter 18 includes thermoelectric cooling elements 52 or RF electrodes 22, the elongate body 46 may include a lumen in electrical communication with an energy generator and/or a power source (not shown).

As shown in FIG. 3, the mapping device 12 and ablation device 18 may be the same device 20, the device 20 including both mapping elements and ablation elements (as also shown in FIGS. 9 and 10). For example, the device 20 may be a catheter including a distal end 48 having an expandable element 14 with a plurality of electrodes 22 coupled thereto. If a single mapping and ablation device 20 is used, the user may adjust the energy generator 40 to increase the energy intensity from mapping to ablation mode and/or may activate cryoablation elements on the target area of scarred tissue 10 that has been mapped. If separate mapping 12 and ablation 18 devices are used, each device 12, 18 may be releasably engageable to the console 34, and may be either be interchangeable or used simultaneously, with control of each device 12, 18 being independent of the other.

Referring now to FIGS. 4 and 5, mapping devices 12 are shown. As described in FIG. 3, the mapping device 12 may be separate from the ablation device 18. The discrete mapping device 12 may be, for example, a focal catheter having a plurality of sensors 16 or electrodes coupled to the distal end (as shown in FIG. 4) or a catheter having an expandable element 14 having a plurality of sensors 16 or electrodes coupled to the expandable element 14 (as shown in FIG. 5). The mapping catheter 12 shown in FIG. 4 includes one or more sensors 16 that detect electrical activity in the heart as the myocardial cells polarize and depolarize (for example, electrodes capable of detecting electrocardiographic measurements). The mapping catheter 12 shown in FIG. 5 includes an expandable element 14, which may either include a protruding distal tip or “nose” 64 (as shown in FIG. 2A) or no protruding distal tip (as shown in FIG. 5). The protruding distal tip 64 may include a tip electrode 66 (for example, as shown in FIG. 8). A balloon having a plurality of sensors 16 coupled to the outer surface is shown, but the expandable element 14 alternatively may be an expandable mesh or basket bearing a plurality of sensors 16. The sensors 16 of mapping catheters in both FIGS. 4 and 5 are electrical conductivity sensors 16, but the mapping catheters may also include other sensors 16 (such as temperature, pressure, and/or magnetic sensors). Further, although shown as circumferential bands in FIG. 4 and longitudinal bands in FIG. 5, any number or arrangement of sensors 16 may be used. A focal catheter may be desirable when mapping small or difficult to navigate areas, whereas a mapping catheter with an expandable element may be desirable when mapping larger, more accessible areas. However, a focal catheter may be deformable at the distal end to assume a variety of shapes, including loops, spirals, or bends, in order to facilitate steering and enhance tissue contact.

Referring now to FIGS. 6, 7, and 8, ablation devices 18 are shown. The ablation device 18 may be a focal catheter (for example, an RFA catheter) having one or more electrodes 22 at the distal end (as shown in FIG. 6), a cryoablation catheter having an expandable element 14 for Joule-Thomson cooling (as shown in FIGS. 3 and 7) or hot balloon ablation, or a catheter with both RF ablation (including PRF) and cryoablation capabilities (as shown in FIG. 8). For example, a focal catheter may be used with phased radiofrequency (PRF) ablation and may be deformable at the distal end 48 to facilitate steering and enhance tissue contact. Additional details related to PRF may be found in U.S. patent application Ser. No. 12/117,596, filed on May 8, 2008, entitled “RF Energy Delivery System and Method,” the entirety of which is hereby incorporated by reference. Further, other ablation devices 18 are contemplated, although not shown, such as a laser, ultrasound, hot balloon, or microwave ablation device. Ablation devices 18 with an expandable element 14 may also include a protruding distal tip 64 (as shown in FIGS. 2B and 8) or no protruding distal tip (as shown in FIG. 7). Still further, the ablation device 18 may be a cryoablation device without an expandable element 14, such as a cryoablation catheter having thermoelectric cooling elements 52 (as shown in FIG. 10).

Referring now to FIGS. 9 and 10, devices 20 are shown that have both mapping and ablation capabilities. As described for FIG. 3, the mapping device 12 and ablation device 18 may be the same device 20, the device 20 including both mapping elements and ablation elements. For example, the device 20 may be a catheter including a distal end 48 having an expandable element 14 with a plurality of sensors 16 and/or electrodes 22 coupled thereto. The expandable element 14 may be a balloon (as shown in FIG. 9) or expandable mesh or basket (not shown). Further, the device may include a protruding distal tip 64 (for example, as shown in FIGS. 2B and 8) or no protruding distal tip (as shown in FIG. 9). The catheter 20 in FIG. 9 includes a plurality of balloon electrodes 22. The balloon electrodes 22 may be distributed in any pattern or arrangement (including a random distribution), but the device 20 shown in FIG. 9 includes electrodes 22 arranged in bands oriented in a distal-to-proximal fashion (longitudinal bands). The electrodes 22 may be strips of conductive material on the outer surface of the balloon 14 or embedded within the walls of the balloon 14, or the electrodes 22 may be part of, for example, a Nitinol mesh on the outer surface of the balloon. Alternatively, the device 20 may be a focal catheter including a distal end 48 having a plurality of electrodes 22 coupled thereto (FIG. 10). The electrodes 22 of the focal catheter of FIG. 10 may have both mapping and ablation capabilities, or may only be used for mapping and a thermoelectric cooling element 52 used for cryoablation. Any number and configuration of electrodes 22 may be used.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A medical system for the ablation of scarred tissue, the medical device comprising:
a medical device including:
an elongate body including a proximal end and a distal end; and
a plurality of electrical conduction sensors coupled to the distal end; and
a console in communication with the distal end of the medical device, the console including a computer,
the computer programmed to identify optimal ablation sites on a target tissue and the location of an isthmus associated with a reentrant VT circuit within a target tissue, the identification being based at least in part on the measurements of the electrical conduction sensors.

19. The medical system of claim 18, wherein the electrical conduction sensors measure at least one of signal amplitudes, relative activation time, activation duration, and monophasic action potentials.

20. (canceled)

21. The medical system of claim 18, wherein the medical device further includes at least one thermoelectric cooling element.

22. The medical system of claim 21, wherein the medical device further includes at least one electrode at the distal end, the at least one electrode being in communication with the at least one thermoelectric cooling element.

23. The medical system of claim 22, wherein the computer is programmable to activate the at least one thermoelectric cooling element when the computer has identified at least one of an optimal ablation site on the target tissue and the location of an isthmus associated with a reentrant VT circuit within the target tissue.

24. A medical system for the ablation of scarred tissue, the medical device comprising:
a medical device including:
an elongate body including a proximal region and a distal region;
a plurality of electrical conduction sensors in the distal region;
at least one thermoelectric cooling element in the distal region; and
at least one area of thermally conductive material in the distal region, the at least one area of thermally conductive material being in communication with the at least one thermoelectric cooling element; and
a console in communication with the plurality of electrical conduction sensors and the at least one thermoelectric cooling element, the console including a computer, the computer being programmed to identify optimal ablation sites on a target tissue and the location of an isthmus associated with a reentrant VT circuit within a target tissue, the identification being based at least in part on the measurements of the electrical conduction sensors, the computer being programmable to activate the at least one thermoelectric cooling element when the computer identifies at least one of an optimal ablation site and the location of an isthmus associated with a reentrant VT circuit, activation of the at least one thermoelectric cooling element causing the at least one area of thermally conductive material to reach ablation temperatures.

25. The medical system of claim 24, wherein the at least one thermoelectric cooling element is distal of the plurality of electrical conduction sensors.

26. The medical system of claim 24, wherein the at least one area of thermally conductive material being distal of the plurality of electrical conduction sensors.
